(19) United States
(12) Patent Application Publication
(10) Pub. No.: US 2012/0253089 A1
(43) Pub. Date: Oct. 4, 2012

(54) PROCESS FOR THE CONVERSION OF LOWER ALKANES TO AROMATIC HYDROCARBONS

(76) Inventors: Mahesh Venkataraman Iyer, Houston, TX (US); Ann Marie Lauritzen, Houston, TX (US); Ajay Madhav Madgavkar, Kary, TX (US)

(21) Appl. No.: 13/505,024

(22) PCT Filed: Oct. 29, 2010
(86) PCT No.: PCT/US10/54599

§ 371 Date: Jun. 13, 2012

Related U.S. Application Data
(60) Provisional application No. 61/257,149, filed on Nov. 2, 2009.

(51) Int. Cl. C07C 2/76

(52) U.S. Cl. 585/300; 585/417

(57) ABSTRACT

The present invention provides a process for producing aromatic hydrocarbons which comprises: (a) alternately contacting a lower alkane feed with an aromatization catalyst under aromatization reaction conditions in a reactor for a short period of time, preferably 30 minutes or less, to produce aromatic reaction products and then contacting the aromatization catalyst with a hydrogen-containing gas at elevated temperature for a short period of time, preferably 10 minutes or less, (b) repeating the cycle of step (a) at least one time, (c) regenerating the aromatization catalyst by contacting it with an oxygen-containing gas at elevated temperature and (d) repeating steps (a) through (c) at least one time.

### FIELD OF THE INVENTION

The present invention relates to a process for producing aromatic hydrocarbons from lower alkanes. More specifically, the invention relates to a process for increasing the productivity of an aromatization catalyst used in a dehydroaromatization process.

### BACKGROUND OF THE INVENTION

There is a projected global shortage for benzene which is needed in the manufacture of key petrochemicals such as styrene, phenol, nylon and polyurethanes, among others. Generally, benzene and other aromatic hydrocarbons are obtained by separating a feedstock fraction which is rich in aromatic compounds, such as reformate produced through a catalytic reforming process and pyrolysis gasolines produced through a naphtha cracking process, from non-aromatic hydrocarbons using a solvent extraction process.

To meet this projected supply shortage, numerous catalysts and processes for on-purpose production of aromatics (including benzene) from alkanes containing six or less carbon atoms per molecule have been investigated. These catalysts are usually bifunctional, containing a zeolite or molecular sieve material to provide acidity and one or more metals such as Pt, Ga, Zn, Mo, etc. to provide dehydrogenation activity. For example, U.S. Pat. No. 4,350,835 describes a process for converting ethane-containing gaseous feeds to aromatics using a crystalline zeolite catalyst of the ZSM-5-type family containing a minor amount of Ga. As another example, U.S. Pat. No. 7,186,871 describes aromatization of C₁-C₄ alkanes using a catalyst containing Pt and ZSM-5.

After a period of time in use during the course of the aromatization reaction, the catalyst becomes deactivated as a result of mechanisms such as the deposition of coke on the catalyst particles. Coke is comprised primarily of carbon, but is also comprised of a small quantity of hydrogen. Coke decreases the ability of the catalyst to promote dehydroaromatization reactions to the point that continued use of the catalyst is no longer practical or economical. At that point, the catalyst must be reconditioned, or regenerated, before it can be reused.

Numerous catalyst regeneration methods are described in the patent literature and nearly all involve to some extent the combustion of coke from the surface of the catalyst. The particular method of regeneration that a specific process employs depends on the design of the catalyst bed(s) in the reactor(s). Fixed catalyst beds keep the catalyst stationary. When the catalyst in a fixed bed reactor becomes deactivated, the reactor is generally temporarily taken out of service while the catalyst is either regenerated in situ or else unloaded and replaced with regenerated or fresh catalyst. Two types of fixed bed regeneration methods are used commercially: cyclic regeneration and semi-regeneration. In the cyclic regeneration method, at least one or at most not all of the reactors are taken out of service at any one time and the process continues in operation with the remaining reactors. After the deactivated catalyst is regenerated, the reactor is placed back in service, which in turn allows another reactor to be taken out of service for regeneration of the catalyst.

Lower alkane aromatization is a highly endothermic reaction that is thermodynamically favored at high temperature and low pressure. Unfortunately, these conditions also facilitate formation of surface coke deposits that deactivate the catalyst relatively rapidly. The coke deposits may be partially or fully removed by subjecting the catalyst to a high-temperature stripping operation with a hydrogen-containing gas stream or steam, or by using an oxygen-containing gas to burn off the accumulated coke. A coke burn is generally preferred for full removal of the accumulated coke deposits, but it must be conducted in a relatively slow, carefully controlled manner to avoid excessive temperature increases that may cause irreversible loss of active catalyst surface area. The useful life of the catalyst is adversely affected if the catalyst is subjected to a large number of high-temperature coke burns between exposures to the lower alkane feed and aromatization conditions.

It would be advantageous to provide a light alkane dehydroaromatization process wherein (a) the deactivation of the catalyst because of coke formation and (b) the adverse effects of high-temperature coke burns can be minimized.

### SUMMARY OF THE INVENTION

The present invention provides a process for producing aromatic hydrocarbons which comprises:

(a) alternately contacting a lower alkane feed with an aromatization catalyst under aromatization reaction conditions in a reactor for a short period of time, preferably about 30 minutes or less, to produce aromatic reaction products and then contacting the aromatization catalyst with a hydrogen-containing gas at elevated temperature for a short period of time, preferably about 30 minutes or less,

(b) repeating the cycle of step (a) at least one time,

(c) regenerating the aromatization catalyst by contacting it with an oxygen-containing gas at elevated temperature,

(d) optionally subjecting the regenerated aromatization catalyst to a metal redispersal treatment,

(e) optionally reducing the regenerated aromatization catalyst, preferably with a hydrogen-containing gas,

(f) optionally sulfiding the catalyst, and

(g) repeating steps (a) through (f) at least one time.

In an embodiment, the process is carried out in at least three reactors, preferably fixed-bed reactors, arranged in parallel and, at any given time, at least one reactor is operated according to step (c) and at least two reactors are operated according to step (a) and in at least one of the at least two reactors operated according to step (a) the aromatization catalyst is contacted with the lower alkane feed and in at least one of the at least two reactors operated according to step (a) the aromatization catalyst is contacted with a hydrogen-containing gas.

In another embodiment, the process is carried out in at least four reactors, preferably fixed-bed reactors, arranged in parallel and, at any given time, at least one reactor is operated according to step (c) and at least three reactors are operated according to step (a) and in at least one of the at least three reactors operated according to step (a) the aromatization catalyst is contacted with the lower alkane feed and in at least one of the at least three reactors operated according to step (a) the aromatization catalyst is contacted with hydrogen.

### BRIEF DESCRIPTION OF THE DRAWINGS

FIG. 1 is a graph which compares the ethane conversion, benzene yield, and total aromatics yield data obtained in Performance Tests 1 and 2 in Example 1.

FIG. 2 is a graph which compares the total (ethane+propane) conversion, benzene yield, and total aromatics yield data obtained in Performance Tests 3 and 4 in Example 2.

FIG. 3 is a graph which compares the ethane conversion, benzene yield, and total aromatics yield data obtained in Performance Tests 5 and 6 in Example 3.

FIG. 4 is a graph which compares the ethane conversion, benzene yield, and total aromatics yield data obtained in Performance Tests 5 and 7 in Example 3.

### DETAILED DESCRIPTION OF THE INVENTION

In the preferred operation/regeneration scheme of the present invention, at any given time a majority of the parallel arranged fixed-bed reactors in a given set are subjected to alternating cycles of (a) short-time (preferably about 30 minutes or less, more preferably about 20 minutes or less, and most preferably about 10 minutes or less, but generally not less than 1 minute) exposure to the lower alkane feed at suitable lower alkane aromatization conditions and (b) short-time (preferably about 30 minutes or less, more preferably about 20 minutes or less, and most preferably about 10 minutes or less, but generally not less than 2 minutes) stripping with a hot hydrogen-containing gas to reheat the catalyst bed and reduce catalyst performance decline by partial removal of surface coke deposits. The timing of this cycling is such that at any given time at least one reactor in the set is exposed to feed and producing aromatics at all times and at least one reactor is exposed to stripping with a hot hydrogen-containing gas at all times. At the same time, at least one of the reactors in the set is completely offline for controlled coke burn regeneration and metal redispersal and/or reduction with a hydrogen-containing gas and/or sulfiding, if needed. Upon completion of the coke burn, the reactor is brought back online for reaction/stripping cycles while another of the parallel arranged reactors, with spent catalyst, is taken offline for coke burn. The pattern continues until all of the reactors have been subjected to coke burn and then repeats. In this way, continuous production of aromatics at high yield is maintained, despite the inherently rapid coking/deactivation of the catalyst under lower alkane aromatization reaction conditions.

The operation/regeneration scheme described above enables continuous production of benzene and other aromatics from cost-advantaged lower alkane feeds at commercially viable rates and yields. This scheme meets the need for frequent catalyst regeneration (coke removal) in a lower alkane aromatization process in a manner that extends the useful operating life of the catalyst or catalysts employed. The alternation of feed exposure and stripping with a hot hydrogen-containing gas in the majority of the parallel reactors at any given time reduces catalyst performance decline over one operational cycle (time between coke burns). This reduction of catalyst performance decline extends the time before a slower, properly-controlled coke burn that will reduce irreversible damage to the catalyst becomes necessary. The useful life of the catalyst is substantially longer when used according to the present invention than if the catalyst is subjected to a higher number of high-temperature coke burns between every exposure to the lower alkane feed and aromatization conditions.

Stripping of coked catalysts with hot hydrogen-containing gas has been practiced commercially for decades and various methods are known to those skilled in the art. The stripping of the catalyst may be carried out in the aromatization reactor. The hydrogen stripping may be carried out by exposing the catalyst to a stream containing up to 100% hydrogen at from about 400 to about 800° C., from about 0.01 to about 1.0 MPa and a weight hourly space velocity (WHSV) of from about 0.1 to about 5 hr⁻¹.

Regeneration of coked catalysts has also been practiced commercially for decades and various regeneration methods are known to those skilled in the art. The regeneration of the catalyst may be carried out in the aromatization reactor. For example, the catalyst may be regenerated by burning the coke at high temperature in the presence of an oxygen-containing gas as described in U.S. Pat. No. 4,795,845 which is herein incorporated by reference in its entirety. The preferred regeneration temperature range for the coke burn regeneration step herein is from about 400 to about 700° C., more preferably from about 400 to about 550° C. The coke burn regeneration method preferred for use herein is to use air or nitrogen-diluted air at about 0.01 to about 1.0 MPa pressure and about 300 to about 2000 gas hourly space velocity (GHSV) feed rate and at a starting temperature nearer to the lower end of the above preferred range which is increased continuously or stepwise to reach a temperature nearer to the upper end of the above preferred range.

The optional metal redispersion step may be carried out by oxychlorination, or by treatment with a solution containing one or more metal redispersing agents, or by various other means known in the art. Metal redispersion methods have been practiced commercially for decades and various methods are known to those skilled in the art. Oxychlorination is preferred for many Pt-containing catalysts, including alumina-supported naphtha reforming catalysts. The steps involved in naphtha reforming catalyst regeneration, including oxychlorination, are described in a review article entitled, “Catalyst Regeneration and Continuous Reforming Issues, by P. K. Doolin, D. J. Zalewski, and S. O. Oyekan, on pages 433-457 of the book Catalytic Naphtha Reforming, 2^{nd}. Edition, edited by G. J. Antos and A. M. Aitani (published by Marcel Dekker, Inc., New York, 2004).

Oxychlorination is preferably carried out with a gas mixture containing water, oxygen, hydrogen chloride and chlorine, and/or one or more organochlorine compounds, such as perchloroethylene, capable of reaction to release chlorine under oxychlorination reaction conditions. Preferably, the oxychorination step is conducted at a temperature ranging from about 480 to about 520° C., with the total concentration of chlorine-containing species in the gas ranging from about 0.01 to 0.6 mol %, the oxygen content of the gas ranging from about 0.1 to about 20 mol % at a partial pressure of up to ca. 25 psia. However, it should be noted, and is well-known to those skilled in the art, that variations in reactor equipment capabilities and metallurgy and/or safety concerns may require upper limits on chlorine compound and/or oxygen content that are substantially lower than those given here in some cases.

The optional reduction step, preferably carried out with hydrogen-containing gas, has been practiced commercially for decades and various methods are known to those skilled in the art including those that use other reducing gases such as carbon monoxide. The reduction serves the purpose of reducing the catalyst metal component to the elemental metallic state and to ensure a relatively uniform dispersion of the metal throughout the support. It may be carried out according to the process described in U.S. Pat. No. 5,106,800, which is herein incorporated by reference in its entirety, specifically by exposing the catalyst to hydrogen-containing gas at a flow rate ranging from about 500 to 6000 GHSV, pressure ranging from about 0.05 to 1.0 MPa, and temperature ranging from about 450 to about 800° C. Sulfiding is another catalyst treatment that has been used for many years in the reactivation of catalysts. It serves the purpose of moderating the catalyst activity to prevent excessive hydrogenolysis and coking reactions. It may be carried out according to the process described in U.S. Pat. No. 5,106,800, which is herein incorporated by reference in its entirety, specifically by treating the reduced catalyst with a sulfiding gas such as a mixture of hydrogen and hydrogen sulfide and/or one or more volatile organosulfur compounds having at least about 10 moles of hydrogen per mole of hydrogen sulfide, more preferably at least 50 moles of hydrogen per mole of sulfur compound(s) at a temperature of from about 200 to about 700° C.

Suitable feed streams for aromatization according to the present invention include alkane streams which may contain primarily one or more C₂, C₃, and/or C₄ alkanes (referred to herein as “lower alkanes”), for example an ethane/propane/butane-rich stream derived from natural gas, refinery or petrochemical streams including waste streams. Examples of potentially suitable feed streams include (but are not limited to) residual ethane and propane from natural gas (methane) purification, pure ethane, propane and butane streams (also known as Natural Gas Liquids) co-produced at a liquefied natural gas site, C₂-C₅ streams from associated gases co-produced with crude oil production, unreacted ethane “waste” streams from steam crackers, and the C₁-C₄ byproduct stream from naphtha reformers. The lower alkane feed may be deliberately diluted with relatively inert gases such as nitrogen and/or with various light hydrocarbons and/or with low levels of additives needed to improve catalyst performance. In one embodiment, the majority of the feedstock is comprised of ethane and propane. In another embodiment, the feedstock is comprised of mixed C₂-C₄ alkanes. In still another embodiment, the feedstock is comprised of primarily propane and/or butane. The feedstock may contain in addition other open chain hydrocarbons containing between 3 and 8 carbon atoms as coreactants. Specific examples of such additional coreactants are propylene, isobutane, n-butenes and isobutene. The feed may contain up to about 20 weight percent of C₂-C₄ olefins, preferably no more than about 10 weight percent olefins. Too much olefin content may cause an unacceptable amount of coking. The hydrocarbon feedstock preferably may be comprised of at least about 30 percent by weight of C₂₋₄ hydrocarbons, preferably at least about 50 percent by weight.

The present invention is a process for producing aromatic hydrocarbons which comprises bringing into contact a hydrocarbon feedstock containing lower alkanes, and possibly other hydrocarbons, and a catalyst composition suitable for promoting the reaction of such hydrocarbons to aromatic hydrocarbons, such as benzene, at a temperature from about 400 to about 700° C. and a pressure from about 0.01 to about 1.0 Mpa absolute. The gas hourly space velocity (GHSV) per hour may range from about 300 to about 6000. The process may be carried out in a single stage or in multiple, preferably two, stages. If a two-stage process is used, the conditions in each stage may fall in the above ranges and may be the same or different.

In one embodiment, the lower alkane feed is comprised of at least about 20% wt propane and about 20% wt ethane and the process is carried out in two stages as described in copending, commonly assigned provisional U.S. Provisional Patent Application 61/257,085 entitled PROCESS FOR THE CONVERSION MIXED LOWER ALKANES TO AROMATIC HYDROCARBONS, filed Nov. 2, 2009, which is herein incorporated by reference in its entirety. In another embodiment, the feed is comprised of at least about 20% wt propane and/or butane and the process is carried out in two stages. The process comprises:

(a) providing a mixed lower alkane feed comprising at least propane and ethane, or at least about 20% wt propane and/or butane, to an aromatization reactor,

(b) alternately contacting the mixed lower alkane feed with a first stage aromatization catalyst in a reactor for a short period of time, preferably about 30 minutes or less, under first stage reaction conditions which maximize the conversion of propane and/or any other higher hydrocarbons present in the feed into first stage aromatic reaction products and then contacting the first stage aromatization catalyst with a hydrogen-containing gas at elevated temperature for a short period of time, preferably about 30 minutes or less,

(c) repeating the cycle of step (b) at least one time,

(d) regenerating the first stage aromatization catalyst by contacting it with an oxygen-containing gas at elevated temperature,

(e) optionally subjecting the regenerated first stage aromatization catalyst to a metal redispersal treatment,

(f) optionally reducing the regenerated first stage aromatization catalyst, preferably with hydrogen-containing gas,

(g) optionally sulfiding the catalyst,

(h) repeating steps (a) through (g) at least one time,

(i) separating the first aromatic reaction products from unreacted and/or byproduct ethane,

(j) alternately contacting unreacted and/or byproduct ethane from step (i) with a second stage aromatization catalyst in a reactor for a short period of time, preferably about 30 minutes or less, under second stage reaction conditions which maximize the conversion of ethane into second stage aromatic reaction products and then contacting the second stage aromatization catalyst with a hydrogen-containing gas at elevated temperature for a short period of time, preferably about 30 minutes or less,

(k) repeating the cycle of step (j) at least one time,

(l) regenerating the second stage aromatization catalyst by contacting it with an oxygen-containing gas at elevated temperature,

(m) optionally subjecting the regenerated second stage aromatization catalyst to a metal redispersal treatment,

(n) optionally reducing the regenerated second stage aromatization catalyst, preferably with hydrogen-containing gas,

(o) optionally sulfiding the catalyst, and

(p) repeating steps (j) through (o) at least one time.

In the first stage, the reaction temperature preferably ranges from about 400 to about 650° C., most preferably from about 420 to about 650° C., and in the second stage, the reaction temperature preferably ranges from about 450 to about 680° C., most preferably from about 450 to about 660° C. The primary desired products of the process of this embodiment are benzene, toluene and/or xylene (BTX). In an embodiment, the first stage reaction conditions may be optimized for the conversion of propane and/or butane to aromatics. Optionally, the first stage reaction conditions may also be optimized for the conversion to aromatics of any higher hydrocarbons which may be present in the feedstock. In another embodiment, the second stage reaction conditions may be optimized for the conversion of ethane to aromatics. Optionally, the second stage reaction conditions may also be optimized for the conversion to BTX of any other non-aromatic hydrocarbons which may be produced in the first stage.

Any one of a variety of catalysts may be used to promote the reaction of the lower alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP 0 244 162. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in U.S. application Ser. No. 12/371,787, filed Feb. 16, 2009 entitled “Process for the Conversion of Ethane to Aromatic Hydrocarbons.” This application is hereby incorporated by reference in its entirety. This application describes a catalyst comprising: (1) 0.005 to 0.1% wt (% by weight) platinum, based on the metal, preferably 0.01 to 0.05% wt, (2) an amount of an attenuating metal selected from the group consisting of tin, lead, and germanium, which is no more than 0.02% wt more than the amount of platinum, preferably not more than 0.2% wt of the catalyst, based on the metal; (3) 10 to 99.9% wt of an aluminosilicate, preferably a zeolite, based on the aluminosilicate, preferably 30 to 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in U.S. Provisional Application No. 61/029,939, filed Feb. 20, 2008 entitled “Process for the Conversion of Ethane to Aromatic Hydrocarbons.” This application is hereby incorporated by reference in its entirety. The application describes a catalyst comprising: (1) 0.005 to 0.1% wt (% by weight) platinum, based on the metal, preferably 0.01 to 0.06% wt, most preferably 0.01 to 0.05% wt, (2) an amount of iron which is equal to or greater than the amount of the platinum but not more than 0.50% wt of the catalyst, preferably not more than 0.20% wt of the catalyst, most preferably not more than 0.10% wt of the catalyst, based on the metal; (3) 10 to 99.9% wt of an aluminosilicate, preferably a zeolite, based on the aluminosilicate, preferably 30 to 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in U.S. application Ser. No. 12/371,803, filed Feb. 16, 2009 entitled “Process for the Conversion of Ethane to Aromatic Hydrocarbons.” This application is hereby incorporated by reference in its entirety. This application describes a catalyst comprising: (1) 0.005 to 0.1 wt % (% by weight) platinum, based on the metal, preferably 0.01 to 0.05% wt, most preferably 0.02 to 0.05% wt, (2) an amount of gallium which is equal to or greater than the amount of the platinum, preferably no more than 1 wt %, most preferably no more than 0.5 wt %, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

The unreacted methane and byproduct hydrocarbons may be used in other steps, stored and/or recycled. It may be necessary to cool these byproducts to liquefy them. When the ethane or mixed lower alkanes originate from an LNG plant as a result of the purification of the natural gas, at least some of these byproducts may be cooled and liquefied using the heat exchangers used to liquefy the purified natural gas (methane).

The toluene and xylene may be converted into benzene by hydrodealkylation. The hydrodealkylation reaction involves the reaction of toluene, xylenes, ethylbenzene, and higher aromatics with hydrogen to strip alkyl groups from the aromatic ring to produce additional benzene and light ends including methane and ethane which are separated from the benzene. This step substantially increases the overall yield of benzene and thus is highly advantageous.

Both thermal and catalytic hydrodealkylation processes are known in the art. Methods for hydrodealkylation are described in US Published Patent Application No. 2009/0156870 which is herein incorporated by reference in its entirety.

The integrated process of this invention may also include the reaction of benzene with propylene to produce cumene which may in turn be converted into phenol and/or acetone. The propylene may be produced separately in a propane dehydrogenation unit or may come from olefin cracker process vent streams or other sources. Methods for the reaction of benzene with propylene to produce cumene are described in US Published Patent Application No. 2009/0156870 which is herein incorporated by reference in its entirety.

The integrated process of this invention may also include the reaction of benzene with olefins such as ethylene. The ethylene may be produced separately in an ethane dehydrogenation unit or may come from olefin cracker process vent streams or other sources. Ethylbenzene is an organic chemical compound which is an aromatic hydrocarbon. Its major use is in the petrochemical industry as an intermediate compound for the production of styrene, which in turn is used for making polystyrene, a commonly used plastic material. Methods for the reaction of benzene with ethylene to produce ethylbenzene are described in US Published Patent Application No. 2009/0156870 which is herein incorporated by reference in its entirety.

Styrene may then be produced by dehydrogenating the ethylbenzene. One process for producing styrene is described in U.S. Pat. No. 4,857,498 which is herein incorporated by reference in its entirety. Another process for producing styrene is described in U.S. Pat. No. 7,276,636, which is herein incorporated by reference in its entirety.

### EXAMPLES

The following examples are provided for illustrative purposes only and are not intended to limit the scope of the invention.

### Example 1

This example illustrates one aspect of the lower alkane aromatization process operating/catalyst regeneration scheme of the present invention. Specifically, this example shows a reduction in catalyst performance decline and coke formation obtainable by operating the process with rapid cycling between hydrocarbon feed exposure and hot hydrogen stripping steps, as opposed to continuous exposure to the hydrocarbon feed. The hydrocarbon feed used for aromatization in this example consists of 100% ethane.

Catalyst A was made on 1.6 mm diameter cylindrical extrudate particles containing 80% wt of zeolite ZSM-5 CBV 3014E powder (30:1 molar SiO₂/Al₂O₃ ratio, available from Zeolyst International) and 20% wt gamma-alumina binder. The extrudate samples were calcined in air up to 650° C. to remove residual moisture prior to use in catalyst preparation. The target metal loadings for Catalyst A were 0.025% w Pt and 0.09% wt Ga.

Metals were deposited on 25-100 gram samples of the above ZSM-5/alumina extrudate by first combining appropriate amounts of stock aqueous solutions of tetraammine platinum nitrate and gallium(III) nitrate, diluting this mixture with deionized water to a volume just sufficient to fill the pores of the extrudate, and impregnating the extrudate with this solution at room temperature and atmospheric pressure. Impregnated samples were aged at room temperature for 2-3 hours and then dried overnight at 100° C.

Samples of Catalyst A, prepared as described above, were tested “as is,” without crushing, in Performance Tests 1 and 2. For each performance test, a 15-cc charge of fresh (not previously tested) catalyst was loaded into a quartz tube (1.40 cm inner diameter) and positioned in a three-zone furnace connected to an automated gas flow system.

Prior to each performance test, the catalyst charge was pretreated in situ at atmospheric pressure (approximately 0.1 MPa absolute) in the following manner:

(a) calcination with air at approximately 60 liters per hour (L/hr), during which the reactor wall temperature was raised from 25 to 510° C. in 12 hours, held at 510° C. for 4 hours, then further increased from 510° C. to 621° C. in 1 hour, then held at 621° C. for 30 minutes;

(b) nitrogen purge at approximately 60 L/hr, 621° C., for 20 minutes; and

(c) reduction with hydrogen at 60 L/hr, 621° C., for 30 minutes.

For Performance Test 1, at the end of the above pretreatment, the hydrogen flow to the reactor was terminated and the catalyst charge was continuously exposed to 100% ethane feed at atmospheric pressure (ca. 0.1 MPa absolute), 621° C. reactor wall temperature, and a feed rate of 1000 GHSV (1000 cc feed per cc of catalyst per hour), for a total of 13 hours.

To monitor changes in catalyst performance during the above test, the total reactor outlet stream was sampled and analyzed by an online gas chromatographic analyzer system. The first online sample was taken ten minutes after introduction of the ethane feed. Subsequent samples were taken every 70 minutes thereafter, for a total of 12 samples during the test. Based on the composition data obtained from the gas chromatographic analysis, ethane conversion was calculated according to the following formula: % ethane conversion=100−% wt ethane in outlet stream. Yields per pass of benzene and total aromatics were given by the % wt amounts of benzene and total aromatics, respectively, in the reactor outlet stream.

At the end of this 13 hour test, the ethane flow to the reactor was terminated and hydrogen was re-introduced at a flow rate of 60 L/hr. The reactor furnace heaters were turned off and the catalyst was allowed to cool to ca. 38° C. over a period of approximately 8 hours.

For Performance Test 2, at the end of the pretreatment described above the catalyst charge was subjected to 157 cycles of alternating exposure to ethane feed and hydrogen at atmospheric pressure (ca. 0.1 MPa) and 621° C. reactor wall temperature according to the following protocol:

(a) 5 minutes of 100% ethane feed at 1000 GHSV

(b) 10 minutes of 100% hydrogen at 4000 GHSV.

The total cumulative exposure time of the catalyst to ethane feed under this test regime was 13.3 hours. The total runtime for the 157 ethane feed/hydrogen stripping cycles described above was 39.9 hours.

To monitor changes in catalyst performance during Performance Test 2, the total reactor outlet stream was sampled and analyzed near the end of selected 5 minute ethane exposure intervals by an online gas chromatographic analyzer system. Ethane conversion, benzene yield per pass, and total aromatics yield per pass were determined in the same manner as for Performance Test 1 above.

At the end of this test, the ethane flow to the reactor was terminated and hydrogen was re-introduced at a flow rate of 60 L/hr. The reactor furnace heaters were turned off and the catalyst was allowed to cool to ca. 38° C. over a period of approximately 8 hours.

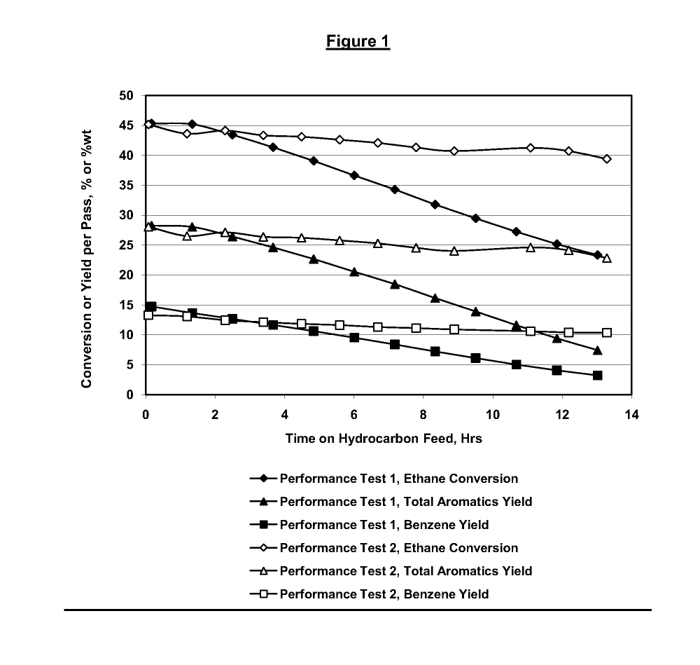

The ethane conversion, benzene yield, and total aromatics yield data obtained in Performance Tests 1 and 2 are compared in FIG. 1. As shown in this figure, the losses in ethane conversion level, benzene yield and total aromatics yield exhibited by the catalyst were much greater during 13 hrs of continuous exposure to ethane feed (Performance Test 1) than during 13.3 hours of cumulative ethane feed exposure under the cyclic feed/hydrogen operating regime used in Performance Test 2. Consistent with these results, the coke (carbon) levels determined by ASTM Method D5291 on the spent catalyst samples from Performance Tests 1 and 2 were 12.2% wt and 7.6% wt, respectively.

### Example 2

This example illustrates one aspect of the lower alkane aromatization process operating/catalyst regeneration scheme of the present invention. Specifically, this example shows a reduction in catalyst performance decline and coke formation obtainable by operating the process with rapid cycling between hydrocarbon feed exposure and hot hydrogen stripping steps, as opposed to continuous exposure to the hydrocarbon feed. The hydrocarbon feed used for aromatization in this example consists of 50% wt ethane and 50% wt propane.

Catalyst B was made on 1.6 mm diameter cylindrical extrudate particles containing 80% wt of zeolite ZSM-5 CBV 2314 powder (23:1 molar SiO₂/Al₂O₃ ratio, available from Zeolyst International) and 20% wt gamma-alumina binder. The extrudate samples were calcined in air up to 650° C. to remove residual moisture prior to use in catalyst preparation. The target metal loadings for Catalyst B were 0.025% w Pt and 0.09% wt Ga.

Samples of Catalyst B, prepared as described above, were tested “as is,” without crushing, in Performance Tests 3 and 4. For each performance test, a 15-cc charge of fresh (not previously tested) catalyst was loaded into a quartz tube (1.40 cm inner diameter) and positioned in a three-zone furnace connected to an automated gas flow system.

Prior to each performance test, the catalyst charge was pretreated in situ at atmospheric pressure (approximately 0.1 MPa absolute) in the following manner:

(a) calcination with air at approximately 60 liters per hour (L/hr), during which the reactor wall temperature was raised from 25 to 510° C. in 12 hours, held at 510° C. for 4 hours, then further increased from 510° C. to 600° C. in 1 hour, then held at 600° C. for 30 minutes;

(b) nitrogen purge at approximately 60 L/hr, 600° C., for 20 minutes;

(c) reduction with hydrogen at 60 L/hr, 600° C., for 30 minutes.

For Performance Test 3, at the end of the above pretreatment, the hydrogen flow to the reactor was terminated and the catalyst charge was continuously exposed to a feed consisting of 50% wt ethane plus 50% wt propane at atmospheric pressure (ca. 0.1 MPa absolute), 600° C. reactor wall temperature, and a feed rate of 1000 GHSV (1000 cc feed per cc of catalyst per hour), for a total of 26 hours.

To monitor changes in catalyst performance during the above test, the total reactor outlet stream was sampled and analyzed by an online gas chromatographic analyzer system.

The first online sample was taken ten minutes after introduction of the ethane/propane feed. Subsequent samples were taken at selected intervals thereafter for the remainder of the test.

Based on the reactor outlet composition data obtained from the gas chromatographic analysis, hydrocarbon feed conversion levels were calculated according to the following formulas:

Ethane conversion, %=100×(% wt ethane in feed−% wt ethane in outlet stream)/(% wt ethane in feed)

Propane conversion, %=100×(% wt propane in feed−% wt propane in outlet stream)/(% wt propane in feed)

Total ethane+propane conversion=((% wt ethane in feed×% ethane conversion)+(% wt propane in feed×% propane conversion))/100

At the end of this test, the ethane/propane feed flow to the reactor was terminated and hydrogen was re-introduced at a flow rate of 60 L/hr. The reactor furnace heaters were turned off and the catalyst was allowed to cool to ca. 38° C. over a period of approximately 8 hours.

For Performance Test 4, at the end of the pretreatment described above, the catalyst charge was subjected to 155 cycles of alternating exposure to 50/50 (w/w) ethane/propane feed and hydrogen at atmospheric pressure (ca. 0.1 MPa) and 600° C. reactor wall temperature according to the following protocol:

(a) 10 minutes of ethane/propane feed at 1000 GHSV

(b) 20 minutes of 100% hydrogen at 4000 GHSV. The total cumulative exposure time of the catalyst to ethane feed under this test regime was 26 hours. The total runtime for the 155 cycles of ethane/propane feed exposure and hydrogen stripping described above was 78 hours.

To monitor changes in catalyst performance during Performance Test 4, the total reactor outlet stream was sampled and analyzed near the end of selected 10 minute ethane/propane exposure cycles by an online gas chromatographic analyzer system. Ethane conversion, propane conversion, total hydrocarbon feed conversion, benzene yield per pass, and total aromatics yield per pass were determined in the same manner as for Performance Test 3 above.

At the end of this test, the ethane/propane feed flow to the reactor was terminated and hydrogen was re-introduced at a flow rate of 60 L/hr. The reactor furnace heaters were turned off and the catalyst was allowed to cool to ca. 38° C. over a period of approximately 8 hours.

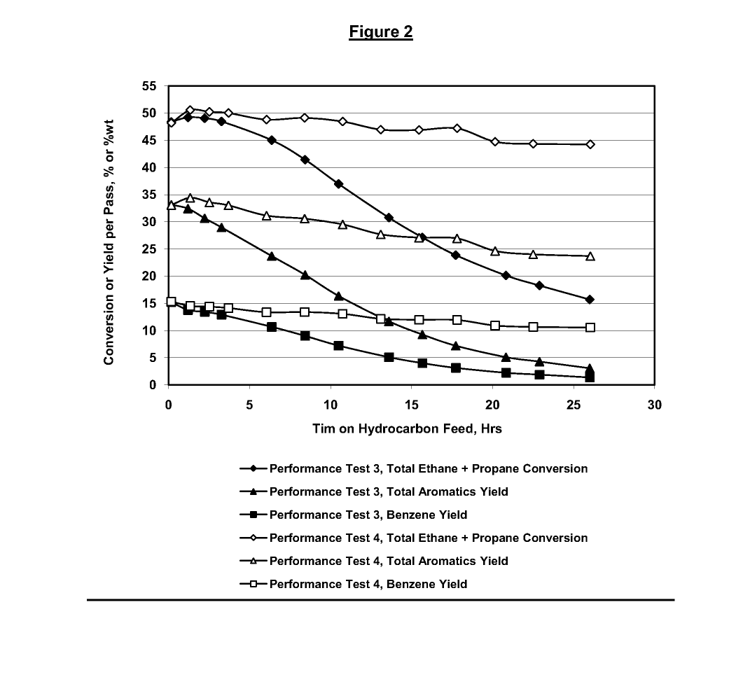

The total feed conversion, benzene yield, and total aromatics yield data obtained in Performance Tests 3 and 4 are compared in FIG. 2. As shown in this figure, the losses in feed conversion level, benzene yield, and total aromatics yield exhibited by the catalyst were much greater during 26 hours of continuous exposure to the hydrocarbon feed (Performance Test 3) than during 26 hours of cumulative hydrocarbon feed exposure under the cyclic feed/hydrogen operating regime used in Performance Test 4. Consistent with these results, the coke (carbon) levels determined by ASTM Method D5291 on the spent catalyst samples from Performance Tests 3 and 4 were 13.9% wt and 8.3% wt, respectively.

### Example 3

In this example, a single catalyst charge is taken through successive tests involving a hydrocarbon feed exposure/hydrogen stripping regime (as described in Examples 1 and 2) and catalyst regeneration procedures involving coke burnoff alone or coke burnoff followed by an oxychlorination treatment. This example illustrates possible operational sequences that could be employed in a single lower alkane aromatization reactor in the process of the present invention. The hydrocarbon feed used for aromatization in this example was 100% ethane.

In Performance Test 5, a fresh 15-cc charge of Catalyst A (see Example 1) was tested with rapid cycling between 100% ethane feed and hydrogen stripping under the same conditions and in the same manner as Performance Test 2 described above in Example 1. Total cumulative exposure time to ethane feed was 13.3 hours and the total runtime was 39.9 hours. At the end of this test, the ethane flow to the reactor was terminated, hydrogen was re-introduced at a flow rate of 60 L/hr, and the reactor wall temperature was lowered from 621° C. to ca. 204° C. in 5 hours. The reactor was then purged with nitrogen at atmospheric pressure (ca. 0.1 MPa) at a flow rate of 60 L/hr for 20 minutes, in preparation for a coke burnoff operation using air.

After the nitrogen purge step, the reactor feed was changed to 10 L/hr air at atmospheric pressure. The reactor wall temperature was then raised from ca. 204° C. to 427° C. in 5 hours, held at 427° C. for 1.5 hours, raised from 427° C. to 482° C. in 1 hour, held at 482° C. for 1.5 hours, raised from 482° C. to 510° C. in 1 hour, held at 510° C. for 4 hours, and then the reactor was allowed to cool to ambient temperature.

Performance Test 6 was conducted in the same manner as Performance Test 5, using the spent, coke-burned charge of Catalyst A from Performance Test 5. At the conclusion of Performance Test 6, the catalyst charge was subjected to a second coke burnoff in air according to the same procedure as that employed at the end of Performance Test 5.

After this second coke burnoff, the spent Catalyst A charge was subjected to an oxychlorination treatment. For this treatment, the 15-cc charge of spent catalyst was loaded into a quartz tube (1.40 cm inner diameter) and positioned in a three-zone furnace and connected to a gas flow system. Nitrogen flow of 30 L/hr was established at atmospheric pressure (ca. 0.1 MPa) and the catalyst was heated from room temperature to 500° C. in 2 hours. When the 500° C. temperature was reached, the gas flowing through the catalyst bed at atmospheric pressure was switched from 30 L/hr nitrogen to 30 L/hr of a gas mixture with the following compositional range: ca. 1.8-2.0% mol oxygen, ca. 1.8-2.0% mol water, ca. 0.8-1.0% mol hydrogen chloride, ca. 0.2-0.3% mol chlorine, balance nitrogen. After 3 hours of exposure to this flowing gas mixture, the gas flowing over the catalyst was switched to 30 L/hr of a mixture consisting of ca. 1.8-2.0% mol oxygen, 1.8-2.0% mol water, balance nitrogen, for 3 hours. At the end of this 3 hour period, the gas flowing over the catalyst was switched to 30 L/hr or air at atmospheric pressure and the catalyst bed was cooled to ambient temperature.

Performance Test 7 was conducted in the same manner as Performance Test 5, using the 15-cc charge of Catalyst A that had been subjected to the oxychlorination treatment described above.

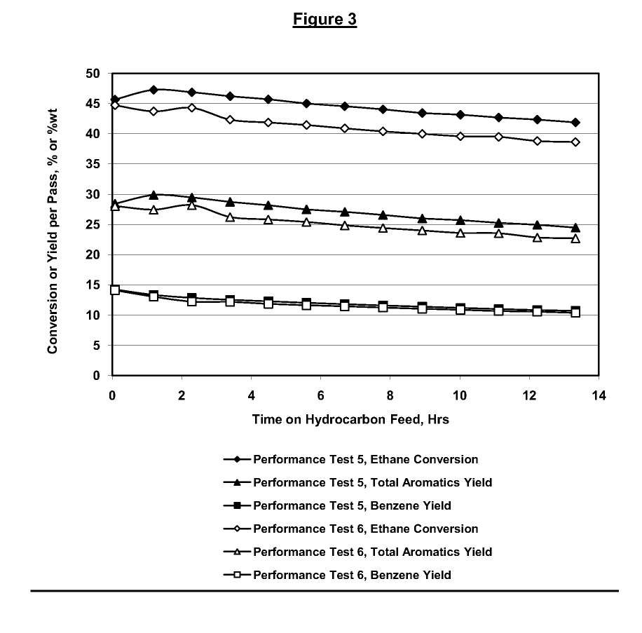

The ethane conversion, total aromatics yield and benzene yield data obtained in Performance Tests 5 and 6 are compared in FIG. 3. The average ethane conversion and total aromatics yield levels displayed by the regenerated catalyst in Performance Test 6 were about 93% of the corresponding values for the fresh catalyst charge in Performance Test 5. The average benzene yield level displayed by the regenerated catalyst in Performance Test 6 was about 97% of the corresponding value for the fresh catalyst in Performance Test 5.

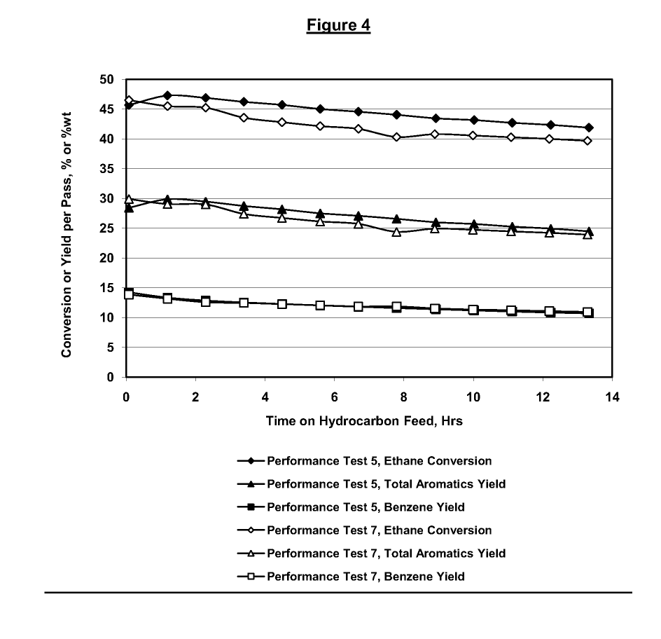

The ethane conversion, total aromatics yield and benzene yield data obtained in Performance Tests 5 and 7 are compared in FIG. 4. The average ethane conversion level displayed by the regenerated catalyst in Performance Test 7 was about 95% of the corresponding value for the fresh catalyst charge in Performance Test 5. The average total aromatics and benzene yields given by the regenerated catalyst in Performance Test 7 were about 97 and 100%, respectively, of the corresponding values for the fresh catalyst in Performance Test 5.

### Example 4

Based on the data from Examples 1 and 3 above, this example outlines a possible scheme for operation of a lower alkane aromatization process using multiple parallel fixed-bed reactors according to the present invention.

The hydrocarbon feed used for aromatization in this example is 100% ethane. In this example, five parallel fixed-bed reactors are operated in cycles lasting approximately 60 hours each. During each 60 hour cycle, each individual reactor operates in the following two modes:

(a) ca. 36 hours in “feed/H₂” mode, in which the catalyst is subjected to rapid cycles of hydrocarbon feed (ca. 5 min) and hydrogen (ca. 10 min) as described for Performance Test 2 (see Example 1);

(b) ca. 24 hours in “regen” mode, in which the catalyst undergoes coke burnoff (such as that described in Example 3), an optional oxychlorination or other metal redispersal step (such as that described in Example 3), and (if needed) a short reduction step with hydrogen in preparation for being brought back online in “feed/H₂” mode.

The timing of each individual reactor's 60 hour operational cycle is staggered so that, during any 12 hour period in the overall 60 hour cycle, three of the five parallel reactors are in “feed/H₂” operational mode, while the other two reactors are in “regen” mode. This staggered timing scheme for a five-reactor system is shown in Table 1 below.

During each 12 hour period in the overall 60 hour cycle, the timing of the feed exposure and hydrogen stripping steps in each of the three online (non-regenerating) reactors is staggered so that during any 15 minute period in the 12 hour interval, one reactor is on hydrocarbon feed producing benzene and other aromatics while the other two reactors are subjected to the hydrogen stripping treatment. This staggered timing scheme for the three parallel online reactors during each 15 minute interval is shown in Table 2.

With the staggered cyclic operating scheme summarized in Tables 1 and 2, aromatics production from a lower alkane feed can occur continuously over a fresh or recently-regenerated catalyst while still meeting the need for frequent catalyst regeneration to maintain overall performance.

### Example 5

Based on the data from Examples 2 and 3 above, this example outlines a possible scheme for operation of a lower alkane aromatization process using multiple parallel fixed-bed reactors according to the present invention.

The hydrocarbon feed used for aromatization in this example consists of 50% wt ethane and 50% wt propane. In this example, four parallel fixed-bed reactors are operated in cycles lasting approximately 96 hours (4 days) each. During each 4 day cycle, each individual reactor operates in the following two modes:

(a) ca. 3 days (72 hours) in “feed/H₂” mode, in which the catalyst is subjected to rapid cycles of hydrocarbon feed (ca. 10 min) and hydrogen (ca. 20 min) as described for Performance Test 4 (see Example 2);

(b) ca. 1 day (24 hours) in “regen” mode, in which the catalyst undergoes coke burnoff (such as that described in Example 3), an optional oxychlorination or other metal redispersal step (such as that described in Example 3), and (if needed) a short reduction step with hydrogen in preparation for being brought back online in “feed/H₂” mode.

The timing of each individual reactor's 4 day operational cycle is staggered so that, during any 1 day period in the overall 4 day cycle, three of the four parallel reactors are in “feed/H₂” operational mode, while the other reactor is in “regen” mode. This staggered timing scheme for a four-reactor system is shown in Table 3.

During each 24 hour period in the overall 96 hr cycle, the timing of the feed exposure and hydrogen stripping steps in each of the three online (non-regenerating) reactors is staggered so that, during any 30 minute period in the 24 hour interval, one reactor is on hydrocarbon feed producing benzene and other aromatics, while the other two reactors are subjected to the hydrogen stripping treatment. This staggered timing scheme for the three parallel online reactors during each 30 minute interval is shown in Table 4.

With the staggered cyclic operating scheme summarized in Tables 3 and 4, aromatics production from a mixed lower alkane feed can occur continuously over a fresh or recently-regenerated catalyst while still meeting the need for frequent catalyst regeneration to maintain overall performance.

TABLE 1

| | TIME IN 60-HR CYCLE | | | | |
|---|---|---|---|---|---|
| | | 12-24 | 24-36 | 36-48 | 48-60 |
| | 0-12 HRS | HRS | HRS | HRS | HRS |
| REACTOR | FEED/H₂ | FEED/H₂ | FEED/H₂ | REGEN | REGEN |
| 1 MODE | | | | | |
| REACTOR | REGEN | FEED/H₂ | FEED/H₂ | FEED/H₂ | REGEN |
| 2 MODE | | | | | |
| REACTOR | REGEN | REGEN | FEED/H₂ | FEED/H₂ | FEED/H₂ |
| 3 MODE | | | | | |
| REACTOR | FEED/H₂ | REGEN | REGEN | FEED/H₂ | FEED/H₂ |
| 4 MODE | | | | | |
| REACTOR | FEED/H₂ | FEED/H₂ | REGEN | REGEN | FEED/H₂ |
| 5 MODE | | | | | |

TABLE 2

TIME IN 15-MIN FEED/H₂ CYCLE

| | 0-5 MIN | 5-10 MIN | 10-15 MIN |
|---|---|---|---|
| REACTOR 1 MODE | FEED | H₂ | H₂ |
| REACTOR 2 MODE | H₂ | FEED | H₂ |
| REACTOR 3 MODE | H₂ | H₂ | FEED |

TABLE 3

| | TIME IN 96-HR CYCLE | | | |
|---|---|---|---|---|
| | | | 48-72 | 72-96 |
| | 0-24 HRS | 24-48 HRS | HRS | HRS |
| REACTOR 1 MODE | FEED/H₂ | FEED/H₂ | FEED/H₂ | REGEN |
| REACTOR 2 MODE | FEED/H₂ | FEED/H₂ | REGEN | FEED/H₂ |
| REACTOR 3 MODE | FEED/H₂ | REGEN | FEED/H₂ | FEED/H₂ |
| REACTOR 4 MODE | REGEN | FEED/H₂ | FEED/H₂ | FEED/H₂ |

TABLE 4

| | TIME IN 30-MIN FEED/H₂ CYCLE | | |
|---|---|---|---|
| | 0-10 MIN | 10-20 MIN | 20-30 MIN |
| REACTOR 1 MODE | FEED | H₂ | H₂ |
| REACTOR 2 MODE | H₂ | FEED | H₂ |
| REACTOR 3 MODE | H₂ | H₂ | FEED |

1. A process for producing aromatic hydrocarbons which comprises:
(a) alternately contacting a lower alkane feed with an aromatization catalyst under aromatization reaction conditions in a reactor for a period of time of 30 minutes or less, to produce aromatic reaction products and then contacting the aromatization catalyst with a hydrogen-containing gas at elevated temperature for a period of time of 30 minutes or less,
(b) repeating the cycle of step (a) at least one time,
(c) regenerating the aromatization catalyst by contacting it with an oxygen-containing gas at elevated temperature,
(d) optionally subjecting the regenerated aromatization catalyst to a metal redispersal treatment,
(e) optionally reducing the regenerated aromatization catalyst, preferably with hydrogen-containing gas,
(f) optionally sulfiding the catalyst, and
(g) repeating steps (a) through (f) at least one time.

2. The process of claim 1 wherein the process is carried out in at least three reactors arranged in parallel and at least one reactor is operated according to step (c) and at least two reactors are operated according to step (a) and in at least one of the at least two reactors operated according to step (a) the aromatization catalyst is contacted with the lower alkane feed and in at least one of the at least two reactors operated according to step (a) the aromatization catalyst is contacted with hydrogen.

3. The process of claims 1 wherein the process is carried out in at least four reactors arranged in parallel and at least one reactor is operated according to step (c) and at least three reactors are operated according to step (a) and in at least one of the at least three reactors operated according to step (a) the aromatization catalyst is contacted with the lower alkane feed and in at least one of the at least three reactors operated according to step (a) the aromatization catalyst is contacted with hydrogen-containing gas.

4. The process of claims 1 wherein step (a) is carried out at 400 to 700° C., 0.01 to 1.0 MPa and a gas hourly space velocity of 300 to 6000 hr⁻¹.

5. The process of claims 1 wherein step (c) is carried out at 400 to 700° C.

6. The process of claims 1 wherein the oxygen-containing gas in step (c) is air.

7. The process of claims 1 wherein after step (c) the regenerated catalyst is subjected to metal redispersal, preferably by oxychlorination.

8. The process of claims 1 wherein after step (c) the regenerated catalyst is reduced, with hydrogen-containing gas.

9. The process of claims 1 wherein after step (c) the regenerated catalyst is sulfided.

10. A process for producing aromatic hydrocarbons which comprises:
(a) providing a lower alkane feed comprising at least propane and ethane, or propane and/or butane,
(b) alternately contacting the lower alkane feed with a first stage aromatization catalyst in a reactor for a period of time of 30 minutes or less, under first stage reaction conditions which maximize the conversion of propane and/or any other higher hydrocarbons present in the feed into first stage aromatic reaction products and then contacting the first stage aromatization catalyst with a hydrogen-containing gas at elevated temperature for a period of time of 30 minutes or less, or less,
(c) repeating the cycle of step (b) at least one time,
(d) regenerating the first stage aromatization catalyst by contacting it with an oxygen-containing gas at elevated temperature,
(e) optionally subjecting the regenerated first stage aromatization catalyst to a metal redispersal treatment,
(f) optionally reducing the regenerated first stage aromatization catalyst, preferably with hydrogen-containing gas,
(g) optionally sulfiding the catalyst,
(h) repeating steps (a) through (g) at least one time,
(i) separating the first aromatic reaction products from unreacted and/or byproduct ethane,
(j) alternately contacting unreacted and/or byproduct ethane from step (i) with a second stage aromatization catalyst in a reactor for a period of time of 30 minutes or less, under second stage reaction conditions which maximize the conversion of ethane into second stage aromatic reaction products and then contacting the second stage aromatization catalyst with a hydrogen-containing gas at elevated temperature for a period of time of 30 minutes or less,
(k) repeating the cycle of step (j) at least one time,
(l) regenerating the second stage aromatization catalyst by contacting it with an oxygen-containing gas at elevated temperature,
(m) optionally subjecting the regenerated second stage aromatization catalyst to a metal redispersal treatment,
(n) optionally reducing the regenerated second stage aromatization catalyst, preferably with a hydrogen-containing gas,
(o) optionally sulfiding the catalyst, and
(p) repeating steps (j) through (o) at least one time.
